(19) United States
(12) Patent Application Publication
(10) Pub. No.: US 2010/0268251 A1
(43) Pub. Date: Oct. 21, 2010

(54) DELIVERY WIRE FOR OCCLUSIVE DEVICE DELIVERY SYSTEM AND METHOD OF MANUFACTURE

(75) Inventors: Hancun Chen, Fremont, CA (US); Jimmy Dao, San Jose, CA (US); Richard Murphy, Sunnyvale, CA (US)

Correspondence Address: David T. Burse;Vista IP Law Group LLP, Suite D-2, 12930 Saratoga Avenue, Saratoga, CA, 95070, US

(73) Assignee: BOSTON SCIENTIFIC SCIMED, INC., Maple Grove, MN (US)

(21) Appl. No.: 12/757,886

(22) Filed: Apr. 9, 2010

Related U.S. Application Data
(60) Provisional application No. 61/170,033, filed on Apr. 16, 2009.

(51) Int. Cl. A61B 17/12; B23P 11/00

(52) U.S. Cl. 606/139; 29/428

(57) ABSTRACT

A delivery wire assembly for delivery of an occlusive device to a location in a patient's vasculature includes a delivery wire conduit having a proximal tubular portion coupled to a distal coil portion, the respective tubular and coil portions defining a conduit lumen, a plug at least partially seated in the conduit lumen and coupled to an interior surface of the coil portion so as to form a substantially fluid tight seal of the conduit lumen, and a core wire disposed in the conduit lumen, the core wire having a distal end extending through the plug and coupled to an occlusive device

### RELATED APPLICATION DATA

The present application claims the benefit under 35 U.S.C. §119 to U.S. provisional patent application Ser. No. 61/170,033, filed Apr. 16, 2009. The foregoing application is hereby incorporated by reference into the present application in its entirety.

### FIELD OF THE INVENTION

The field of the invention generally relates to systems and delivery devices for implanting vaso-occlusive devices for establishing an embolus or vascular occlusion in a vessel of a human or veterinary patient.

### BACKGROUND OF THE INVENTION

Vaso-occlusive devices or implants are used for a wide variety of reasons, including treatment of intra-vascular aneurysms. Commonly used vaso-occlusive devices include soft, helically wound coils formed by winding a platinum (or platinum alloy) wire strand about a “primary” mandrel. The relative stiffness of the coil will depend, among other things, on its composition, the diameter of the wire strand, the diameter of the primary mandrel, and the pitch of the resulting primary windings. The coil is then wrapped around a larger, “secondary” mandrel, and heat treated to impart a secondary shape. For example, U.S. Pat. No. 4,994,069, issued to Ritchart et al., describes a vaso-occlusive coil that assumes a linear, helical primary shape when stretched for placement through the lumen of a delivery catheter, and a folded, convoluted secondary shape when released from the delivery catheter and deposited in the vasculature.

In order to deliver the vaso-occlusive coils to a desired site in the vasculature, e.g., within an aneurismal sac, it is well-known to first position a small profile, delivery catheter or “micro-catheter” at the site using a steerable guidewire. Typically, the distal end of the micro-catheter is provided, either by the attending physician or by the manufacturer, with a selected pre-shaped bend, e.g., 45°, 90°, “J”, “S”, or other bending shape, depending on the particular anatomy of the patient, so that it will stay in a desired position for releasing one or more vaso-occlusive coil(s) into the aneurysm once the guidewire is withdrawn. A delivery or “pusher” wire is then passed through the micro-catheter, until a vaso-occlusive coil coupled to a distal end of the delivery wire is extended out of the distal end opening of the micro-catheter and into the aneurysm. The vaso-occlusive device is then released or “detached” from the end delivery wire, and the delivery wire is withdrawn back through the catheter. Depending on the particular needs of the patient, one or more additional occlusive devices may be pushed through the catheter and released at the same site.

One well-known way to release a vaso-occlusive coil from the end of the pusher wire is through the use of an electrolytically severable junction, which is a small exposed section or detachment zone located along a distal end portion of the pusher wire. The detachment zone is typically made of stainless steel and is located just proximal of the vaso-occlusive device. An electrolytically severable junction is susceptible to electrolysis and disintegrates when the pusher wire is electrically charged in the presence of an ionic solution, such as blood or other bodily fluids. Thus, once the detachment zone exits out of the catheter distal end and is exposed in the vessel blood pool of the patient, a current applied through an electrical contact to the conductive pusher wire completes a circuit with a return electrode, and the detachment zone disintegrates due to electrolysis. Return electrodes include electrodes attached to the patient's skin, conductive needles inserted through the skin at a remote site, and electrodes located on the pusher wire but electrically insulated from the conductive path ending in the detachment zone. The anode is made up of an insulated core wire, which runs through the pusher wire, is attached to the electrical contact at the proximal end, and forms the detachment zone at the distal end.

Perceived problems with current vaso-occlusive coil delivery systems include electrical shorts or current leakage in the electrolytic detachment system. The electrical insulation surrounding the core wire may have imperfections that lead to two types of shorts. Current leakage (a wet short) occurs when body fluid leaks into the pusher wire and makes contact with the core wire exposed by the imperfections in the insulation. An intermittent or direct hard short (a dry short) occurs when the exposed core wire makes direct contact with the inside of the pusher wire. Current leakage and electrical shorts may adversely impact detachment of the embolic coil by electrolysis.

### SUMMARY

In one embodiment, a delivery wire assembly for delivery of an occlusive device to a location in a patient's vasculature includes a delivery wire conduit, a plug, and a core wire. The delivery wire conduit has a proximal tubular portion coupled to a distal coil portion, which together define a conduit lumen. The plug is at least partially seated in the conduit lumen and coupled to an interior surface of the coil portion so as to form a substantially fluid tight seal of the conduit lumen. The core wire is disposed in the conduit lumen, and has a distal end extending through the plug and coupled to an occlusive device. The plug may include a polymer tube attached to one or both of the core wire and the interior surface of the coil portion via a friction fit. The plug may also include a stopper coil attached to one or both of the core wire and the interior surface of the coil portion with an adhesive. The stopper coil may be attached to the core wire with a non-conductive adhesive and attached to the delivery wire conduit with a conductive adhesive. The stopper coil may extend partially out of a distal opening of the conduit lumen. The occlusive device may be attached to the core wire via an electrolytically severable junction. The stopper coil, the proximal tubular portion, and the distal coil portion may form a conductive path for current dissolving the junction when the device is in situ. A sleeve may be disposed around at least a portion of the delivery wire conduit. The sleeve may be secured to the delivery wire conduit by heat lamination.

In another embodiment, an occlusive device delivery system includes a delivery catheter and a delivery wire assembly seated in the delivery catheter. The delivery catheter includes a proximal end, a distal end, and a catheter lumen extending between the proximal and distal ends. The delivery wire assembly includes a delivery wire conduit, a plug, and a core wire. The delivery wire conduit has a proximal tubular portion coupled to a distal coil portion, which together define a conduit lumen. The plug is at least partially seated in the conduit lumen and coupled to an interior surface of the coil portion so as to form a substantially fluid tight seal of the conduit lumen. The core wire is disposed in the conduit lumen, and has a distal end extending through the plug and coupled to an occlusive device.

In yet another embodiment, a method of manufacturing a delivery wire assembly for delivery of an occlusive device to a location in a patient's vasculature includes the steps of providing a wire and a long body, connecting the wire to the long body, and inserting the long body into a delivery wire conduit. The method also includes the steps of providing sufficient tension to the wire to straighten the wire, sliding the delivery wire conduit from the long body over the wire, cutting the wire on both ends of the delivery wire conduit, and adding an electrical contact and a plug to the proximal and distal ends of the delivery wire conduit, respectively.

In still another embodiment, a method of manufacturing a delivery wire assembly for delivery of an occlusive device to a location in a patient's vasculature includes the steps of providing a wire and a delivery wire conduit having a long body disposed therein, connecting the wire to the long body and providing sufficient tension to the wire to straighten the wire. The method also includes the steps of sliding the delivery wire conduit from the long body over the wire, cutting the wire on both ends of the delivery wire conduit, and adding an electrical contact and a plug to the proximal and distal ends of the delivery wire conduit, respectively.

### BRIEF DESCRIPTION OF THE DRAWINGS

Referring now to the drawings in which like reference numbers represent corresponding parts throughout, and in which:

FIG. 1 illustrates an occlusive coil delivery system, according to one embodiment.

FIG. 2 is a longitudinal cross-sectional view of a delivery wire assembly, according to one embodiment.

FIG. 3 illustrates an occlusive coil in a natural state mode, illustrating one exemplary secondary configuration.

FIGS. 4A to 4D are detailed longitudinal cross-sectional views of delivery wire assemblies, according to various embodiments.

FIG. 5 is a schematic diagram showing the steps in the manufacture of a delivery wire assembly, according to one embodiment.

FIG. 6 is a schematic diagram showing the steps in the manufacture of a delivery wire assembly, according to another embodiment.

### DETAILED DESCRIPTION OF THE ILLUSTRATED EMBODIMENTS

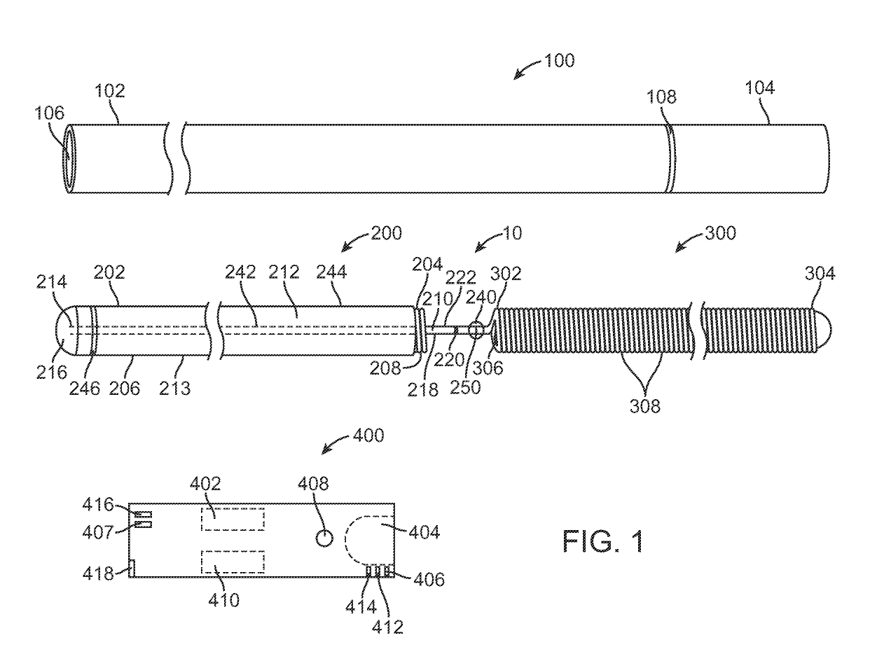

FIG. 1 illustrates an occlusive coil delivery system 10 according to one embodiment. The system 10 includes a number of subcomponents or sub-systems. These include a delivery catheter 100, a delivery wire assembly 200, an occlusive coil 300, and a power supply 400. The delivery catheter 100 includes a proximal end 102, a distal end 104, and a lumen 106 extending between the proximal and distal ends 102, 104. The lumen 106 of the delivery catheter 100 is sized to accommodate axial movement of the delivery wire assembly 200. Further, the lumen 106 is sized for the passage of a guidewire (not shown) which may optionally be used to properly guide the delivery catheter 100 to the appropriate delivery site.

The delivery catheter 100 may include a braided-shaft construction of stainless steel flat wire that is encapsulated or surrounded by a polymer coating. For example, HYDROLENE® is one exemplary polymer coating that may be used to cover the exterior portion of the delivery catheter 100. Of course, the system 10 is not limited to a particular construction or type of delivery catheter 100 and other constructions known to those skilled in the art may be used for the delivery catheter 100.

The inner lumen 106 is advantageously coated with a lubricious coating such as PTFE to reduce frictional forces between the delivery catheter 100 and the device that is being moved axially within the lumen 106. The delivery catheter 100 may include one or more optional marker bands 108 formed from a radiopaque material that can be used to identify the location of the delivery catheter 100 within the patient's vasculature system using imaging technology (e.g., fluoroscope imaging). The length of the delivery catheter 100 may vary depending on the particular application but generally is around 150 cm in length. Of course, other lengths of the delivery catheter 100 may be used with the system 10 described herein.

The delivery catheter 100 may include a distal end 104 that is straight as illustrated in FIG. 1. Alternatively, the distal end 104 may be pre-shaped into a specific geometry or orientation. For example, the distal end 104 may be shaped into a “C” shape, an “S” shape, a “J” shape, a 45° bend, a 90° bend. The size of the lumen 106 may vary depending on the size of the delivery wire assembly 200 and occlusive coil 300 but generally the diameter of the lumen 106 of the delivery catheter 100 (I.D. of delivery catheter 100) is less than about 0.02 inches. The delivery catheter 100 is known to those skilled in the art as a microcatheter. While not illustrated in FIG. 1, the delivery catheter 100 may be utilized with a separate guide catheter (not shown) that aids in guiding the delivery catheter 100 to the appropriate location within the patient's vasculature.

Still referring to FIG. 1, the system 10 includes a delivery wire assembly 200 that is configured for axial movement within the lumen 106 of the delivery catheter 100. The delivery wire assembly 200 generally includes a proximal end 202 and a distal end 204. The delivery wire assembly 200 includes a delivery wire conduit 213, which has a proximal tubular portion 206 and a distal coil portion 208. The proximal tubular portion 206 may be formed from, for example, stainless steel hypotube. The distal coil portion 208 may be formed from, for example, stainless steel wire. The distal coil portion 208 may be bonded to the proximal tubular portion 206 in an end-to-end arrangement.

The delivery wire assembly 200 further includes a core wire 210 that extends from the proximal end 202 of the delivery wire assembly 200 to a location that is distal with respect to the distal end 204 of the delivery wire assembly 200. The core wire 210 is disposed within a lumen 212 that extends within an interior portion of the delivery wire conduit 213. The core wire 210 is formed from an electrically conductive material such as stainless steel wire. The proximal end 214 of the core wire 210 (shown in phantom) is electrically coupled to an electrical contact 216 located at the proximal end 202 of the delivery wire assembly 200. The electrical contact 216 may be formed from a metallic solder (e.g., gold) that is configured to interface with a corresponding electrical contact (not shown) in the power supply 400.

A portion of the core wire 210 is advantageously coated with an insulative coating 218. The insulative coating 218 may include polyimide. The entire length of the core wire 210 is coated with an insulative coating 218 except for the proximal end 214 of the core wire 210 that is in contact with electrical contact 216 and a small region 220 located in a portion of the core wire 210 that extends distally with respect to the distal end 204 of the of the delivery wire assembly 200. This latter “bare” portion of the core wire 210 forms the electrolytic detachment zone 220 which dissolves upon application of electrical current from the power supply 400.

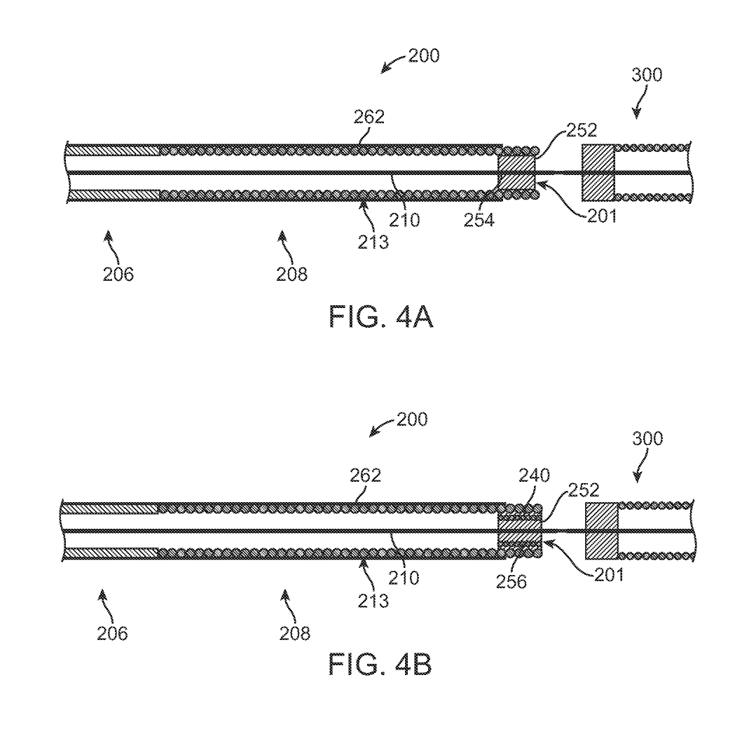

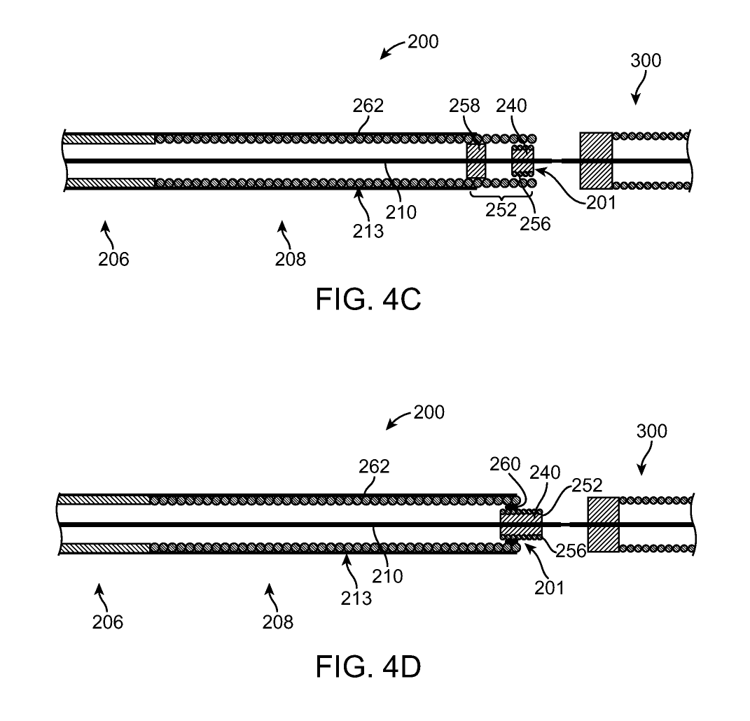

Still referring to FIG. 1, the occlusive coil 300 includes a proximal end 302, a distal end 304 and a lumen 306 extending there between. The occlusive coil 300 is generally made from a biocompatible metal such as platinum or a platinum alloy (e.g., platinum-tungsten alloy). The occlusive coil 300 generally includes a straight configuration (as illustrated in FIG. 1) when the occlusive coil 300 is loaded within the delivery catheter 100. Upon release, the occlusive coil 300 generally takes a secondary shape which may include two-dimensional or three-dimensional configurations such as that illustrated in FIG. 4. Of course, the system 10 described herein may be used with occlusive coils 300 having a variety of configurations and is not limited to particular occlusive coils 300 having a certain size or configuration.

The occlusive coil 300 includes a plurality of coil windings 308. The coil windings 308 are generally helical about a central axis disposed along the lumen 306 of the occlusive coil 300. The occlusive coil 300 may have a closed pitch configuration as illustrated in FIG. 1.

The distal end 222 of the core wire 210 is connected to the proximal end 302 of the occlusive coil 300 at a junction 250. Various techniques and devices can be used to connect the core wire 210 to the occlusive coil 300, including laser melting, and laser tack, spot, and continuous welding. It is preferable to apply an adhesive 240 to cover the junction 250 formed between the distal end 222 of the core wire 210 and the proximal end 302 of the occlusion coil 300. The adhesive 240 may include an epoxy material which is cured or hardened through the application of heat or UV radiation. For example, the adhesive 240 may include a thermally cured, two-part epoxy such as EPO-TEK® 353ND-4 available from Epoxy Technology, Inc., 14 Fortune Drive, Billerica, Mass. The adhesive 240 encapsulates the junction 250 and increases its mechanical stability.

Still referring to FIG. 1, the system 10 includes a power supply 400 for supplying direct current to the core wire 210 which contains the electrolytic detachment zone 220. In the presence of an electrically conductive fluid (which may include a physiological fluid such as blood or a flushing solution such as saline), when the power supply 400 is activated, electrical current flows in a circuit including the electrical contact 216, the core wire 210, the electrolytic detachment zone 220, and a return electrode (not shown). After several seconds (generally less than about 10 seconds), the sacrificial electrolytic detachment zone 220 dissolves and the occlusive coil 300 separates form the core wire 210.

The power supply 400 will include an onboard energy source such as batteries (e.g., a pair of AAA batteries) along with drive circuitry 402. The drive circuitry 402 may include one or more microcontrollers or processors configured to output a driving current. The power supply 400 illustrated in FIG. 1 includes a receptacle 404 that is configured to receive and mate with the proximal end 202 of the delivery wire assembly 200. Upon insertion of the proximal end 202 into the receptacle 404, the electrical contact 216 disposed on the delivery wire assembly 200 electrically couple with corresponding contacts (not shown) located in the power supply 400.

A visual indicator 406 (e.g., LED light) may indicate when the proximal end 202 of delivery wire assembly 200 has been properly inserted into the power supply 400. Another visual indicator 407 may activate if the batteries need to be replaced. The power supply 400 typically includes an activation trigger or button 408 that is depressed by the user to apply the electrical current to the sacrificial electrolytic detachment zone 220. Typically, once the activation trigger 408 has been activated, the driver circuitry 402 automatically supplies current until detachment occurs. The drive circuitry 402 typically operates by applying a substantially constant current (e.g., around 1.5 mA).

The power supply 400 may include optional detection circuitry 410 that is configured to detect when the occlusive coil 300 has detached from the core wire 210. The detection circuitry 410 may identify detachment based upon a measured impedance value. A visual indicator 412 may indicate when the power supply 400 is being supplied to the current to the sacrificial electrolytic detachment zone 220. Another visual indicator 414 may indicate when the occlusive coil 300 has detached from the core wire 210. As an alternative to the visual indicator 414, an audible signal (e.g., beep) or even tactile signal (e.g., vibration or buzzer) may be triggered upon detachment. The detection circuitry 410 may be configured to disable the drive circuitry 402 upon sensing detachment of the occlusive coil 300.

The power supply 400 may also contain another visual indicator 416 that indicates to the operator when non-bipolar delivery wire assembly is inserted into the power supply 400. As explained in the background above, non-bipolar delivery wire assemblies use a separate return electrode that typically is in the form of a needle that was inserted into the groin area of the patient. The power supply 400 is configured to detect when a non-bipolar delivery wire assembly has been inserted. Under such situations, the visual indicator 416 (e.g., LED) is turned on and the user is advised to insert the separate return electrode (not shown in FIG. 1) into a port 418 located on the power supply 400.

Still referring to FIG. 1, the core wire 210 forms a first conductive path 242 between the electrical contact 216 and the electrolytic detachment zone 220. This first conductive path 242 may comprise the anode (+) of the electrolytic circuit when the delivery wire assembly 200 is operatively coupled to the power supply 400. A second conductive path 244, the return path, is formed by the proximal tubular portion 206 and a distal coil portion 208 of the delivery wire conduit 213. The second conductive path 244 is electrically isolated from the first conductive path 242. The second conductive path 244 may comprise the cathode (−) or ground electrode for the electrical circuit.

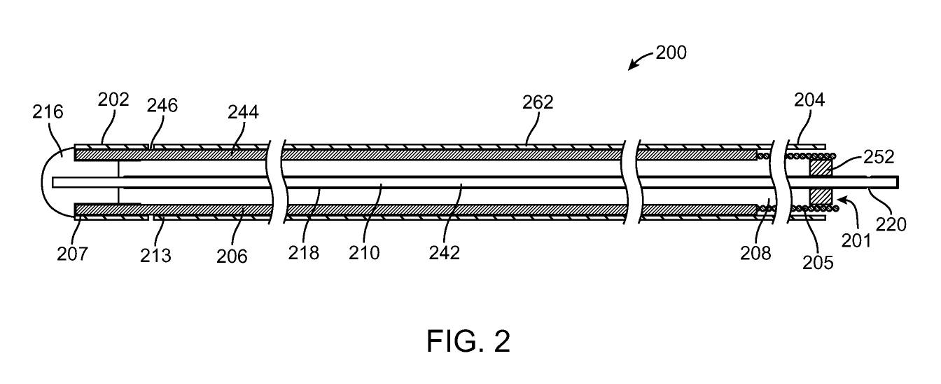

A ground contact 246 for the second conductive path 244 may be disposed on a proximal end of the tubular portion 206 of the delivery wire conduit 213. In one embodiment, the ground contact 246 is simply an exposed portion of the tubular portion 206 since the tubular portion 206 is part of the second conductive path 244. For instance, a proximal portion of the tubular portion 206 that is adjacent to the electrical contact 216 may be covered with an insulative coating 207 such as polyimide as illustrated in FIG. 2. An exposed region of the tubular portion 206 that does not have the insulative coating may form the ground contact 246. Alternatively, the ground contact 246 may be a ring type electrode or other contact that is formed on the exterior of the tubular portion 206.

The ground contact 246 is configured to interface with a corresponding electrical contact (not shown) in the power supply 400 when the proximal end 202 of the delivery wire assembly 200 is inserted into the power supply 400. The ground contact 246 of the second conductive path 244 is, of course, electrically isolated with respect to the electrical contact 216 of the first conductive path 242.

FIG. 2 illustrates a cross-sectional view of the delivery wire assembly 200 according to one embodiment. Similar elements of this embodiment are identified with the same reference numbers as discussed above with respect to FIG. 1. The delivery wire assembly 200 includes a proximal end 202 and a distal end 204 and measures between around 184 cm to around 186 cm in length. The delivery wire assembly 200 includes a delivery wire conduit 213 with a proximal tubular portion 206, a distal coil portion 208, and a distal opening 201. The proximal tubular portion 206 may be formed from stainless steel hypotube having an outer diameter (OD) of 0.01325 inches and inner diameter (ID) of 0.0075 inches. The length of the hypotube section may be between around 140 cm to around 150 cm, although other lengths may also be used.

As seen in FIG. 2, a distal coil portion 208 is bonded in end-to-end fashion to the distal face of the proximal tubular portion 206. The bonding may be accomplished using a weld or other bond. The distal coil portion 208 may have a length of around 39 cm to around 41 cm in length. The distal coil portion 208 may comprise a coil of 0.0025 inches×0.006 inches. The first dimension generally refers to the OD of the coil wire that forms the coil. The latter dimension generally refers to the internal mandrel used to wind the coil wire around to form the plurality of coil winds and is the nominal ID of the coil.

One or more marker coils 205 of the distal coil portion 208 may be formed from a radiopaque material (illustrated as solid marker coils 205 in distal coil portion 208). For example, the distal coil portion 208 may include a segment of stainless steel coil (e.g., 3 cm in length), followed by a segment of platinum coil (which is radiopaque and also 3 mm in length), followed by a segment of stainless steel coil (e.g., 37 cm in length), and so on and so forth.

An outer sleeve 262 or jacket surrounds a portion of the proximal tubular portion 206 and a portion of the distal coil portion 208 of the delivery wire conduit 213. The outer sleeve 262 covers the interface or joint formed between the proximal tubular portion 206 and the distal coil portion 208. The outer sleeve 262 may have a length of around 50 cm to around 54 cm. The outer sleeve 262 may be formed from a polyether block amide plastic material (e.g., PEBAX 7233 lamination). The outer sleeve 262 may include a lamination of PEBAX and HYDROLENE® that may be heat laminated to the delivery wire assembly 200. The OD of the outer sleeve 262 may be less than 0.02 inches and advantageously less than 0.015 inches.

The core wire 210, which runs through the delivery wire conduit 213, terminates at electrical contact 216 at one end and extends distally with respect to the distal coil portion 208 of the delivery wire conduit 213. The core wire 210 is coated with an insulative coating 218 such as polyimide except at the electrolytic detachment zone 220 and the proximal segment coupled to the electrical contact 216. The electrolytic detachment zone 220 is located several centimeters (e.g., about 2 to about 4 cm) distally with respect to the distal end of the distal coil portion 208. The core wire 210 may have an OD of around 0.00175 inches.

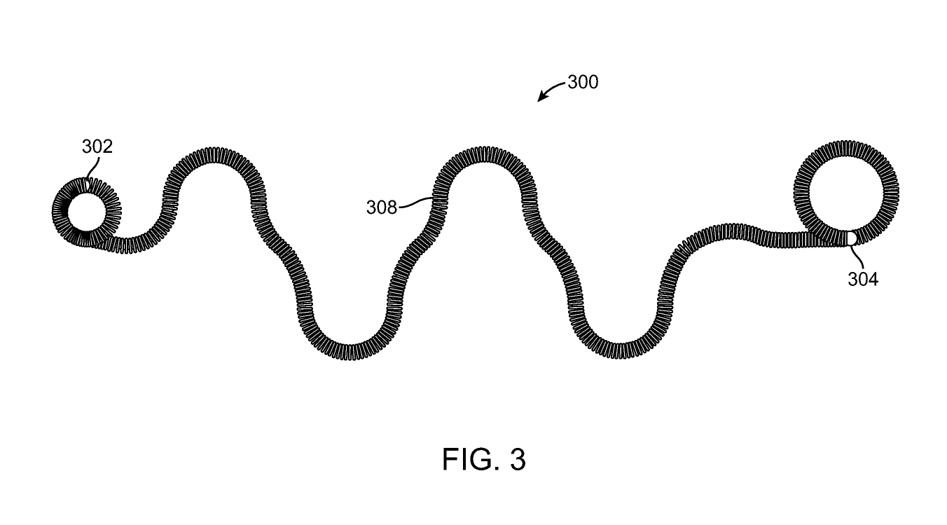

FIG. 3 illustrates one exemplary configuration of an occlusive coil 300 in a natural state. In the natural state, the occlusive coil 300 transforms from the straight configuration illustrated in, for instance, FIG. 1 into a secondary shape. The secondary shaped may include both two and three dimensional shapes of a wide variety. FIG. 3 is just one example of a secondary shape of an occlusive coil 300 and other shapes and configurations are contemplated to fall within the scope of the invention. Also, the occlusive coil 300 may incorporate synthetic fibers over all or a portion of the occlusive coil 300 as is known in the art. These fibers may be attached directly to coil windings 308 or the fibers may be integrated into the occlusive coil 300 using a weave or braided configuration.

As shown in FIG. 4A to 4D, the distal opening 201 of the delivery wire conduit 213 is sealed with a plug 252. In one embodiment, as shown in FIG. 4A, the plug 252 is made of polymer tubing, e.g., PEBAX or Fluorinated Ethylene Propylene (FEP). The opening 254 in the plug 252 is sized to fit the core wire 210, ID of around 0.00225 inches. The relative dimensions of the opening 254 in the plug 252 and the core wire 210 results in a friction fit that holds the plug 252 in position once it has been threaded onto the core wire 210. Alternatively, the plug 252 is held in position by a non-conductive adhesive, which secures it to the core wire 210 and the inside of the delivery wire conduit 213.

In another embodiment, as shown in FIG. 4B, the plug 252 sealing the distal opening 201 of the delivery wire conduit 213 includes a stopper coil 256, which is held in place by non-conductive adhesive 240 connecting it to the core wire 210 and the inside of the delivery wire conduit 213. The adhesive 240 may include EPO-TEK® 353ND-4 described in more detail above. The stopper coil 256 is made of stainless steel wire.

In yet another embodiment, as shown in FIG. 4C, the plug 252 sealing the distal opening 210 of the delivery wire conduit 213 includes both a stopper coil 256 and a segment of polymer tubing 258. The stopper coil 256 is held in place by non-conductive adhesive 240 connecting it to the core wire 210. The segment of polymer tubing 258 is held in place by a friction fit as described above.

In still another embodiment, as shown in FIG. 4D, the plug 252 sealing the distal opening 201 of the delivery wire conduit 213 also includes a stopper coil 256 made of stainless steel wire. The stopper coil 256 is held in place by non-conductive adhesive 240 connecting it to the core wire 210. The stopper coil 256 is also held in place by conductive adhesive 260, i.e., silver epoxy, connecting it to the inside of the delivery wire conduit 213. The conductive adhesive 260 electrically connects the stopper coil 256 to the ground electrode 246 via the delivery wire conduit 213, making it part of the second conductive path 244 described above.

All of the plugs 252 shown in FIGS. 4A to 4D seal the distal opening 201 of the delivery wire conduit 213 to reduce leakage of body fluid into the delivery wire conduit 213. Such body fluid leakage may lead to current leakage and wet shorts. The plugs 252 also ensure that the core wire 210 is properly oriented within the delivery wire assembly 200. In addition, the stopper coil 256 shown in FIG. 4D extends the second conductive path 244 and acts as a return electrode. This eliminates the need to remove the outer sleeve 262 from the distal tip of the distal coil portion 208 of the delivery wire conduit 213 after lamination.

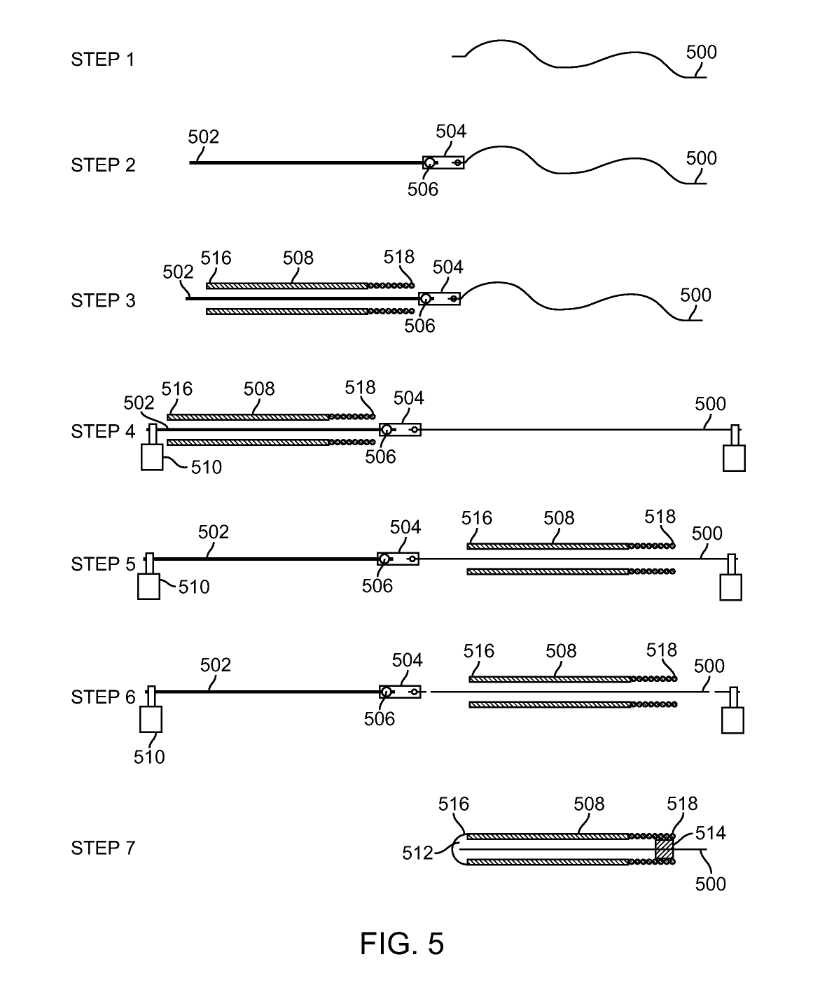

The delivery wire assembly 200 may be manufactured as shown in FIG. 5. In Step 1, a polyimide coated stainless steel wire 500 is provided. Next, in Step 2, a polymer tube 504 and a stainless steel mandrel 502 are provided and the wire 500 and the mandrel 502 are connected to opposite ends of the polymer tube 504 with adhesive 506. In Step 3, the mandrel 502 is threaded through a delivery wire conduit 508, such as the FIRM Coil-Hypotube Assembly. Then in Step 4, the free ends of the wire 500 and the mandrel 502 are connected to clamps 510 and a small tension is supplied to the wire 500—polymer tube 504—mandrel 502 assembly to straighten out the wire 500. In Step 5, the delivery wire conduit 508 is slid from the mandrel 502 to the wire 500. Next, in Step 6, the wire 500 is cut on either side of the delivery wire conduit 508. In Step 7, an electrical contact 512 and a plug 514 are added to the proximal and distal ends 516, 518 of the delivery wire conduit 508. The tension applied during assembly reduces frictional damage to the wire 500 from contact with the inside of the delivery wire conduit 508.

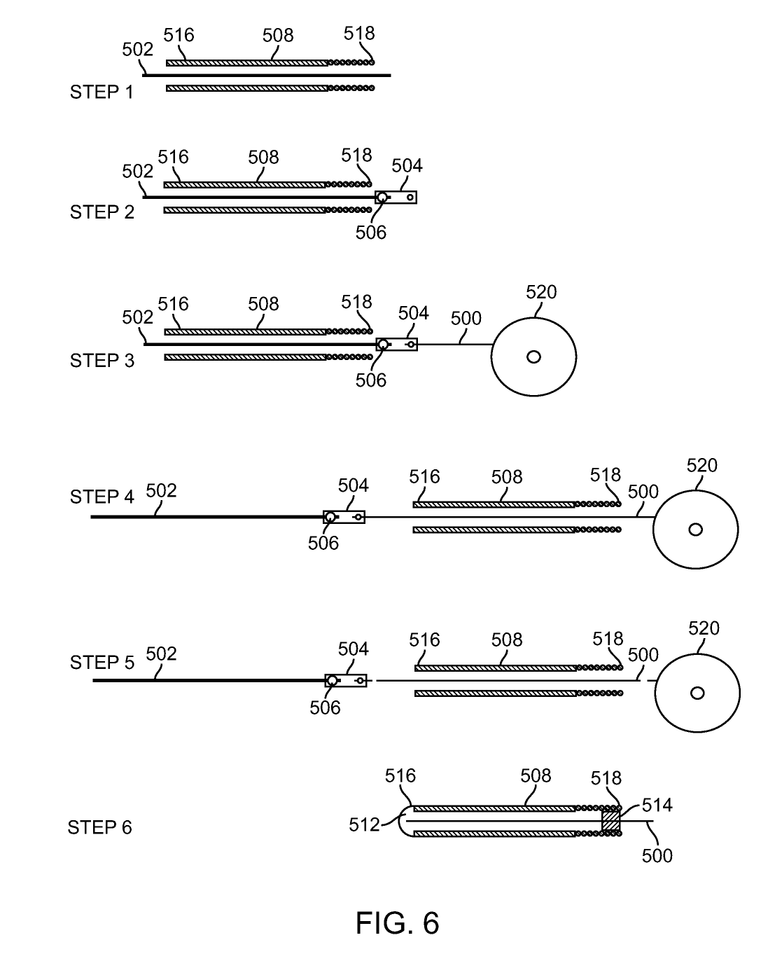

The delivery wire assembly 200 may also be manufactured as shown in FIG. 6. In Step 1, a delivery wire conduit 508, such as the FIRM Coil-Hypotube Assembly having a shipping mandrel 502 is provided and the delivery wire conduit 508 is fixed in place. Next, in Step 2, a polymer tube 504 is provided and the mandrel 502 is connected to one end of the polymer tube 504 with adhesive 506. In Step 3, a coil 520 of polyimide coated stainless steel wire 500 with pre-laser ablated zones is provided and the wire 500 is connected to the other end of the polymer tube 504 with adhesive 506. Then in Step 4, a small tension is supplied to the wire 500—polymer tube 504—mandrel 502 assembly to straighten out the wire 500 and the wire 500 is slowly pulled through the delivery wire conduit 508. In Step 5, the wire 500 is cut on either side of the delivery wire conduit 508. Next, in Step 6, an electrical contact 512 and a plug 514 are added to the proximal and distal ends 516, 518 of the delivery wire conduit 508. The tension applied during assembly reduces frictional damage to the wire 500 from contact with the inside of the delivery wire conduit 508.

The electrical contact 512 may be manufactured by applying a metallic solder to the proximal end 516 of the delivery wire conduit 508 and the wire 500 at that end. After the metallic solder is allowed to cure, clippers or the like may be used to trim the excess material. The plug 514 may be manufactured by sliding a segment of polymer tubing onto the wire 500 at the distal end 518 of the delivery wire conduit 500.

While various embodiments of the present invention have been shown and described, they are presented for purposes of illustration, and not limitation. Various modifications may be made to the illustrated and described embodiments without departing from the scope of the present invention, which is to be limited and defined only by the following claims.

What is claimed is:

1. A delivery wire assembly for delivery of an occlusive device to a location in a patient's vasculature, comprising:
a delivery wire conduit having a proximal tubular portion coupled to a distal coil portion, the respective tubular and coil portions defining a conduit lumen;
a plug at least partially seated in the conduit lumen and coupled to an interior surface of the coil portion so as to form a substantially fluid tight seal of the conduit lumen; and
a core wire disposed in the conduit lumen, the core wire having a distal end extending through the plug and coupled to an occlusive device.

2. The delivery wire assembly of claim 1, wherein the plug comprises a polymer tube.

3. The delivery wire assembly of claim 2, wherein the polymer tube is attached to one or both of the core wire and the interior surface of the coil portion via a friction fit.

4. The delivery wire assembly of claim 1, wherein the plug comprises a stopper coil.

5. The delivery wire assembly of claim 4, wherein the stopper coil is attached to one or both of the core wire and the interior surface of the coil portion with an adhesive.

6. The delivery wire assembly of claim 1, wherein plug comprises a polymer tube attached to the core wire via a friction fit, and a stopper coil attached to the core wire with an adhesive.

7. The delivery wire assembly of claim 4, wherein the stopper coil extends partially out of a distal opening of the conduit lumen.

8. The delivery wire assembly of claim 7, wherein the stopper coil is attached to the core wire with a non-conductive adhesive and the stopper coil is attached to the delivery wire conduit with a conductive adhesive.

9. The delivery wire assembly of claim 8, wherein the occlusive device is attached to the core wire via an electrolytically severable junction, and wherein respective stopper coil, the proximal tubular portion, and the distal coil portion form a conductive path for current dissolving the junction when the device is in situ.

10. The delivery wire assembly of claim 1, further comprising a sleeve disposed around at least a portion of the delivery wire conduit.

11. The delivery wire assembly of claim 10, wherein the sleeve is secured to the delivery wire conduit by heat lamination.

12. An occlusive device delivery system, comprising:
a delivery catheter comprising a proximal end, a distal end, and a catheter lumen extending between the proximal and distal ends; and
a delivery wire assembly seated in the delivery catheter, the delivery wire assembly including
a delivery wire conduit having a proximal tubular portion coupled to a distal coil portion, the respective tubular and coil portions defining a conduit lumen,
a plug at least partially seated in the conduit lumen and coupled to an interior surface of the coil portion so as to form a substantially fluid tight seal of the conduit lumen, and
a core wire disposed in the conduit lumen, the core wire having a distal end extending through the plug and coupled to an occlusive device.

13. A method of manufacturing a delivery wire assembly for delivery of an occlusive device to a location in a patient's vasculature, comprising:
providing a wire and a long body;
connecting the wire to the long body;
inserting the long body into a delivery wire conduit;
providing sufficient tension to the wire to straighten the wire;
sliding the delivery wire conduit from the long body over the wire;
cutting the wire on both ends of the delivery wire conduit; and
adding an electrical contact and a plug to the proximal and distal ends of the delivery wire conduit, respectively.
